(19) United States
(12) Patent Application Publication
(10) Pub. No.: US 2024/0165638 A1
(43) Pub. Date: May 23, 2024

(54) MICRO ELECTROSTATIC PURIFICATION DEVICE FOR RESISTING HIGH HUMIDITY

(71) Applicant: AirQuality Technology (Shanghai) Co., Ltd., Shanghai (CN)

(72) Inventors: Shidan LONG, Shanghai (CN); Shanqing MENG, Shanghai (CN); Huanyu ZHANG, Shanghai (CN); Kejun JIANG, Shanghai (CN); Qin LI, Shanghai (CN)

(21) Appl. No.: 18/424,755

(22) Filed: Jan. 27, 2024

Related U.S. Application Data
(63) Continuation of application No. PCT/CN2022/142361, filed on Dec. 27, 2022.

(30) Foreign Application Priority Data
Oct. 14, 2022 (CN) 202211260929.9

(51) Int. Cl. B03C 3/86; B03C 3/45

(52) U.S. Cl. CPC B03C 3/86; B03C 3/45

(57) ABSTRACT

The present application discloses a micro electrostatic purification device for resisting high humidity, including an ionization module, a micro electrostatic filter and a power supply module, the two ends of the insulating dielectric material in the cartridge carbon electrode length direction are installed inward respectively into the first electrode strip and the second electrode strip, and finally closed with a sealant, and the connection of the individual wire is sealed by filling sealant, to achieve the complete closure of the cartridge. The connection between the power module and the electrode strip, and the connection between the electrode contact piece and the external power supply are all completely sealed through the setting of the structure. And a current limitation protection circuit is provided to monitor the working current in real time and carry out current limitation protection when the instantaneous change of the working current exceeds the threshold.

### CROSS-REFERENCE TO RELATED APPLICATIONS

The present application claims priority to Chinese Patent Application No. 202211260929.9, filed on Oct. 14, 2022, the entire disclosures of which are incorporated herein by reference.

### TECHNICAL FIELD

The present application belongs to the field of air purification technology, and particularly to a micro electrostatic purification device for resisting high humidity.

### BACKGROUND

Micro electrostatic precipitator is a technology that utilises a strong electric field carried by dielectric material for air purification. The dielectric material wraps electrode sheet to form a honeycomb hollow microporous channel, a strong electric field is formed inside the channel, to exert a great attraction to the movement of charged particles in the air, and can collect and kill the bacteria, viruses and other microorganisms attached to the particles in the strong electric field. The micro electrostatic precipitator can not only efficiently remove PM1.0, PM2.5, PM10, but also effectively remove white staphylococcus, natural bacteria, H1N1 Influenza a virus, SARS-CoV-2 new coronavirus and other microorganisms to achieve high efficiency broad-spectrum bacteriostatic disinfection, and has a small resistance, no consumables, safe and harmless, man-machine coexistence, and does not produce hazardous substances (eg, ozone) characteristics, therefore, it is widely used in the purification and disinfection products of human environment.

Micro electrostatic precipitator consists of an ionisation zone (ionisation module) and a dust collection zone (micro electrostatic filter), both of which are powered by a high-voltage power supply, which can either be built into the ionisation module and the micro electrostatic filter or placed outside the ionisation module and the micro electrostatic filter, and connected by wires. The micro electrostatic precipitator, according to the two different typical forms of the ionisation module, is divided into a charge micro electrostatic precipitator by the ion generator to release the charge, and a field electric micro electrostatic precipitator composed of the field electric module constituted by the metal pole plate with the tip. The former is more suitable for use in small spaces with indoor circulation, while the latter is more suitable for use in places where outdoor fresh air is introduced or in medium to large spaces with complex airflow organization.

In high humidity environments, such as >80% relative humidity, the micro electrostatic air purifiers often can not work stably and continuously, and the efficiency, dust capacity with the increase of humidity will be rapidly attenuated. Moreover, under high humidity, once the dielectric material of the micro electrostatic filter inside the purifier is broken, or due to unreasonable structural design and sealing, resulting in increased leakage paths, the micro electrostatic filter will be very easy to generate current leakage, arcing and ignition, which will lead to the inability of the purifier to work normally; and due to the failure of the voltage and current control, it will even cause the dielectric material at the current leakage of micro electrostatic filter to increase in temperature, melt through, and until a large area is burnt. Because such problem cannot be solved, for the micro electrostatic air purifiers in the market, their efficiency is reduced and their dust capacity is attenuated in the high humidity environment and the accident of fire is happened from time to time.

### SUMMARY

The purpose of the present application is to provide a micro electrostatic purification device for resisting high humidity to satisfy applications in high humidity environments.

In order to achieve the above purpose, the technical solution of the present application is as follow:
a micro electrostatic purification device for resisting high humidity, including a filter cartridge,
the filter cartridge is formed by stacking insulating dielectric plates with an air channel between an upper plate and a lower plate of the insulating dielectric plate, and a space keeping isolation member is provided between the upper plate and the lower plate of the insulating dielectric plate, and the space keeping isolation member and the upper plate and the lower plate of the insulating dielectric plate form the air channel;
an electrode material is provided on the insulating dielectric plate, the electrode material is recessed into a preset distance relative to each of four edges of the insulating dielectric plate, and the recessed distance at one end is greater than the recessed distance at another end in a length direction;
when the insulating dielectric plates with the electrode material are stacked, the insulating dielectric plates in an upper layer and a lower layer are stacked horizontally opposite to each other;
edges of the filter cartridge are cut by a hot fusion to completely seal the electrode material;
two ends of the filter cartridge are cut out two electrode strip holding slots in the length direction by the hot fusion for holding a first electrode strip and a second electrode strip respectively, and the first electrode strip and second electrode strip are electrically coupled to the electrode material with a short recessed distance at an end where the first electrode strip or second electrode strip is located, respectively; and
the first electrode strip and the second electrode strip are also connected with insulating wires respectively, the first electrode strip and second electrode strip and a connector connected with the insulating wires are sealed in the electrode strip holding slot of the filter cartridge.

In an embodiment, the device further includes an upper housing and a lower housing for accommodating the filter cartridge.

In an embodiment, another end of the insulating wire connected to the first electrode strip and the second electrode strip is connected to a power supply module, and the power supply module is an external power supply module;
wherein the power supply module includes a sampling and comparison circuit, a current limitation circuit, and a voltage adjustment circuit;
when the sampling and comparison circuit detects a change in an output voltage, the voltage adjustment circuit adjusts the output voltage to a constant output voltage;
when the sampling and comparison circuit detects that a change in a current exceeds a preset threshold, the current limitation circuit limits a load current; and
the micro electrostatic filter further includes a first electrode contact piece and a second electrode contact piece connected to the power supply module, the first electrode contact piece is connected to the first electrode strip through the insulating wire, and the second electrode contact piece is connected to the second electrode strip through the insulating wire.

In an embodiment, the lower housing is provided with a first chamber and a second chamber for accommodating the first electrode contact piece and the second electrode contact piece, respectively, and the first chamber and second chamber are sealed by filling glue after accommodating the first electrode contact piece and the second electrode contact piece.

In an embodiment, each of the first chamber and the second chamber is provided with two semi-projecting separator plates inside, and centers of the first electrode contact piece and the second electrode contact piece are recessed and are stuck between the two semi-projecting separator plates.

In an embodiment, the lower housing is provided with two grooves between the first chamber and the second chamber.

In an embodiment, another end of the insulating wire connected to the first electrode strip and the second electrode strip is connected to a power supply module, and the power supply module is provided inside the upper housing or the lower housing;
wherein the power supply module includes a sampling and comparison circuit, a current limitation circuit, and a voltage adjustment circuit;
when the sampling and comparison circuit detects a change in an output voltage, the voltage adjustment circuit adjusts the output voltage to a constant output voltage;
when the sampling and comparison circuit detects that a change in a current exceeds a preset threshold, the current limitation circuit limits a load current;
an output of the power supply module is connected to the first electrode strip and the second electrode strip through the insulating wire, an input of the power supply module is connected to a third electrode contact piece and a fourth electrode contact piece, the third electrode contact piece and the fourth electrode contact piece are clamped in the chamber opened in the upper housing or the lower housing; and
a joint where the third electrode contact piece and the fourth electrode contact piece are connected with the insulating wire, and a joint where the power supply module is connected with the insulating wire, are sealed by pouring in a sealant.

In an embodiment, magnets are also provided on both sides of the upper housing or the lower housing.

In an embodiment, the air channel of insulating dielectric plates of the filter cartridge are quadrilateral, the quadrilateral air channels are staggered, and the space keeping isolation member of the quadrilateral air channels of one insulating dielectric plate is located between two space keeping isolation members of the quadrilateral air channels of another insulating dielectric plate.

In an embodiment, the electrode material is recessed a preset distance with respect to each of the four edges of the insulating dielectric plate, and the recessed distance at one end in the length direction is greater than the recessed distance at another end, wherein the recessed distance at one end in the length direction is 10-30 mm and the recessed distance at another end is 2-10 mm; and
the recessed distance from the left edge and the right edge is 2-15 mm.

In an embodiment, a distance from a bottom of the electrode strip holding slot to the electrode material with the short recessed distance at the end or extend into the electrode material with the short recessed distance at the end is 0-10 mm, and a width of the bottom of the electrode strip holding slot is 2-10 mm; the two electrode strip holding slots are fit with the first electrode strip and the second electrode strip.

In an embodiment, the raw material of the insulating dielectric plate includes granular insulating dielectric raw material of 89-93% by weight, a flame retardant of 3-8% by weight, and a masterbatch of 1-3% by weight; the granular insulating dielectric raw material is at least one of polyethylene, polypropylene, polyvinyl chloride, polystyrene, acrylonitrile-butadiene-styrene co-polymer, polyformaldehyde, polyamide, polycarbonate, polyethylene terephthalate, or polyimide; the flame retardant is at least one of triisocyanate (2,3-dibromopropyl), tribromophenol, trityl phosphate, trityl phosphite, or dimethyl methyl phosphonate.

In an embodiment, the insulating dielectric plates provided with the electrode materials are stacked and then covered with at least one more layer of insulating dielectric plates not provided with electrode materials on a side where the electrode materials are bare.

The present application proposes a micro electrostatic purification device for resisting high humidity, for the high relative humidity environment, the micro electrostatic air purifier works unstably, with low efficiency and other issues, four aspects such as the frame structure, the sealing of the electrode circuit, the filter structural design and the power supply to limit the current, are improved simultaneously, to improve the reasonableness of the frame structure, sealing of electrode circuits, the filter structural design, and to increase the detection and limitation of the power supply to the current, so that in the high humidity environment, the stability of the work is improved, the work of the efficiency and the amount of dust capacity are ensured, and the current leakage, high humidity, low efficiency, the arcing and ignition, and other issues are avoided.

### BRIEF DESCRIPTION OF THE DRAWINGS

FIG. 1 shows a schematic structural view of a micro electrostatic purification device according to a first embodiment of the present application.

FIG. 2 shows an exploded view of the micro electrostatic purification device according to the first embodiment of the present application.

FIG. 3 shows a schematic diagram of the insulating dielectric plate according to an embodiment of the present application.

FIG. 4 is a comparative schematic diagram of air channels staggered.

FIG. 5 shows a schematic diagram of the stacking of insulating dielectric plates of the present application.

FIG. 6 shows a schematic structural view of the filter cartridge of the present application.

FIG. 7 shows a schematic diagram of an installation chamber of the electrode contact piece according to the embodiment of the present application.

FIG. 8 shows a schematic diagram of a shape of the electrode contact piece according to the embodiment of the present application.

FIG. 9 shows an exploded view of the power supply module provided inside the micro electrostatic filter of the present application.

FIG. 10 is a schematic diagram of installation of the power supply module according to the second embodiment of the present application.

FIG. 11 is a block diagram of the power supply module according to the embodiment of the present application.

### DETAILED DESCRIPTION OF THE EMBODIMENTS

In order to make the purpose, technical solutions and advantages of the present application more clearly understood, the present application is described in further detail hereinafter in conjunction with the accompanying drawings and embodiments. It should be understood that the specific embodiments described herein are only for explaining the present application and are not intended to limit the present application.

The existing micro electrostatic purification device suffers from the following problems in a high humidity environment:
1. the frame of the micro electrostatic purification device and the electrode circuit are not completely sealed, or the surface dielectric material is damaged.

When the electrode circuit of the micro electrostatic purification device is not completely sealed or the surface dielectric material is damaged, in a high humidity environment or even in the presence of water droplets, it is very easy to form a short circuit of the circuit, resulting in current leakage, breakdown, arcing and ignition, and the dielectric material wraps around the electrode circuit nearby will also be broken through, fired, or even burnt; and if there is particulate matter attached to the electrode circuit, it will be more likely to collect the charge on the electrode plate, and the adsorbed particulate matter is also be easier to adsorb liquid droplets in the air, and its bulge is more likely to increase the current leakage, and reduce the efficiency of the filter, and further damage the coated dielectric material, thereby affecting the normal and safe operation.
2. constant power supply open-loop control or electronic control loop has no current limiting design.

In a certain voltage rise interval, the purification efficiency of the micro electrostatic purification device is proportional to the voltage, the higher the voltage, the higher the purification efficiency. With the increase of air humidity, the voltage is too high, it is easy to lead to the arcing and ignition, which will lead to the failure of the micro electrostatic purification device, or produce open flame fire. At present, most of the power supply module of the micro electrostatic purification device is constant power, using open-loop control, that is, the output voltage is changed with the load. As the air humidity increases, the accumulation of dust in the micro electrostatic purification device will cause changes in voltage, resulting in reduced purification efficiency or even failure.

Moreover, the power supply module of the micro electrostatic purification device generally does not carry a detection and control module, and there is no monitoring and limitation of the current, i.e., the output current changes with the load. As the air humidity increases, the air is easier to be ionized or even breakdown, and the accumulation of dust in the micro electrostatic purification device causes the ionisation gap to become smaller, at which time the current increases dramatically and reaches a maximum when it is completely breakdown, resulting in a short circuit of the purifier to be burnt-out, which can seriously cause an open fire.

The following describes a micro electrostatic purification device of the present application for resisting high humidity by means of two embodiments, namely, an embodiment in which the power supply module is external and an embodiment in which the power supply module is provided inside, which are described separately below.

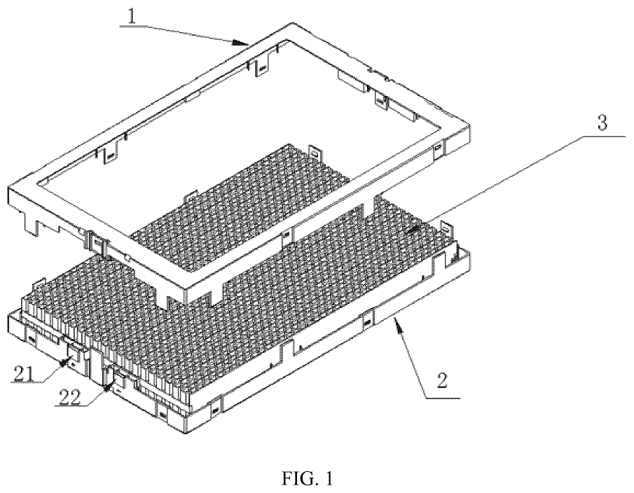

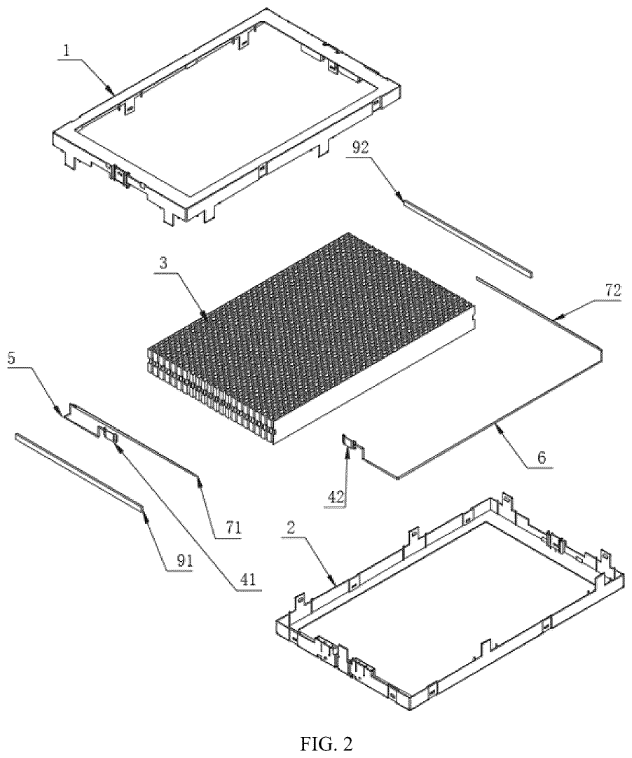

The first embodiment, as shown in FIGS. 1 and 2, the micro electrostatic purification device includes a filter cartridge 3;
the filter cartridge 3 is formed by stacking insulating dielectric plates with an air channel between an upper plate and a lower plate, and a space keeping isolation member 102 is provided between the upper plate and the lower plate of the insulating dielectric plate, and the space keeping isolation member 102 and the upper plate and the lower plate of the insulating dielectric plate form the air channel; an electrode material is provided on the insulating dielectric plate, the electrode material is recessed into a preset distance relative to each of four edges of the insulating dielectric plate, and the recessed distance at one end is greater than the recessed distance at another end in a length direction; when the insulating dielectric plates with the electrode material are stacked, the insulating dielectric plates in a top layer and a bottom layer are stacked horizontally opposite to each other; edges of the filter cartridge 3 are cut by a hot fusion to completely seal the electrode material;
two ends of the filter cartridge 3 are cut out by the hot fusion two electrode strip holding slots in the length direction for holding a first electrode strip 71 and a second electrode strip 72 respectively, and the first electrode strip 71 and the second electrode strip 72 are electrically coupled to the electrode material with a short recessed distance at an end where the first electrode strip 71 or the second electrode strip 72 is located, respectively; and
the first electrode strip 71 and the second electrode strip 72 are also connected with insulating wires respectively, the first electrode strip 71 and second electrode strip 72 and a connector connected with the insulating wires are sealed in the electrode strip holding slot of the filter cartridge 3.

In one specific implementation, the micro electrostatic purification device for resisting high humidity further includes an upper housing 1 and a lower housing 2, the upper housing 1 and lower housing 2 are snapped together to form a space for accommodating the filter cartridge 3.

It is to be noted that the present application is not limited to the specific form of composition of the housing accommodating the filter cartridge, as the embodiment of FIG. 1 embodies only one form, for example, it is also possible to have a box directly accommodating the filter cartridge therein, or to directly employ fasteners at both ends to clamp the filter cartridge for use, and so on.

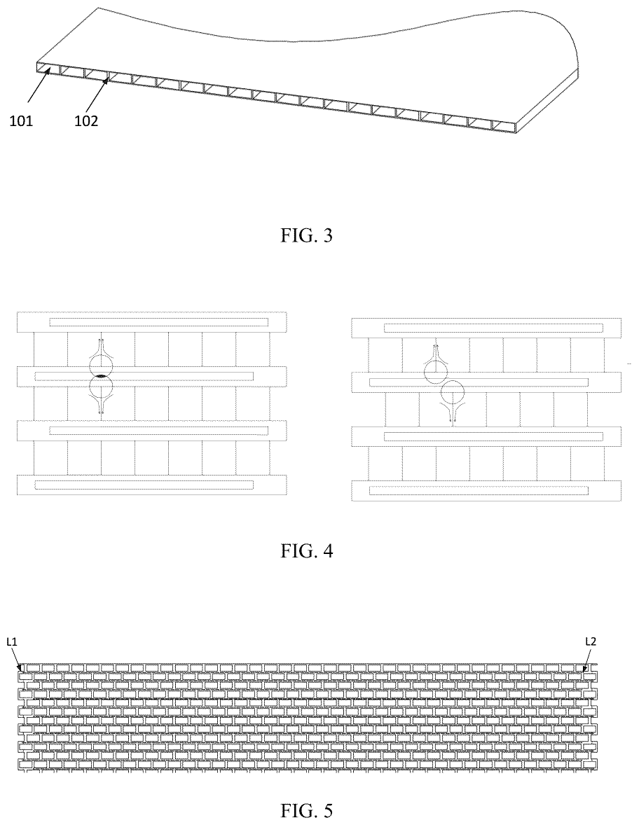

Specifically, the filter cartridge 3 is formed by stacking multiple layers of insulating dielectric plates, as shown in FIG. 3, the single-layer insulating dielectric plate includes the upper plate and the lower plate, and the space keeping isolation member 102 provided between the upper plate and the lower plate, and the space keeping isolation member and the upper plate and the lower plate form the air channel 101. Due to the space keeping isolation member's supporting effect, the shape of the insulating dielectric plate is maintained better, and the spacing of the electrode material on both sides is fixed, so it is not easy to occur the phenomenon of ignition and the corona discharge problem caused by the electrodes not being parallel is avoided.

In the embodiment, the air channel is quadrilateral, but it can also be other shapes such as a triangle, a trapezoid, etc. In a specific embodiment, when the insulating dielectric plates are stacked, the quadrilateral air channels of neighbouring insulating dielectric plates are staggered, as shown in FIG. 4, whereby the space keeping isolation member of the quadrilateral air channel of one insulating dielectric plate is located between two space keeping isolation members of the quadrilateral air channels of another insulating dielectric plate. In the case of the air channels of other shapes are similarly staggered, so that the space keeping isolation members are staggered from each other.

The space keeping isolation member in the left part of FIG. 4 are not staggered, and the corners of air channels in the upper and lower layers are prone to cause breakdown of the electrodes or ignition phenomena after dust collection occurs. Instead, the present application adopts the design of the right part of FIG. 4, whereby the space keeping isolation member of the quadrilateral air channel is located between two space keeping isolation members of the quadrilateral air channels of another insulating dielectric plate, preferably in an intermediate position. Such design makes each corner of the quadrilateral air channels to be staggered, so that when dust collection occurs, a short circuit will not be formed due to the dust collection, and the phenomenon of ignition can be effectively avoided in a high humidity environment. Each strip electrode is parallel and equidistant, avoiding the corona discharge problem caused by electrodes that are not parallel.

When the purification device adsorbs particles, due to the space keeping isolation member and the insulating dielectric plate to form an approximate 90-degree right angle, where friction is generated with the air, particles and other dirt is more likely to be concentrated in this bending corner, the side of the insulating dielectric plate facing the direction of the air flow is also prone to carry particles and other dirt. Therefore, in the later operation, these two places will be more likely to accumulate dirt, that is, the resistance value of the leakage path decreases, and the leakage current is likely to increase.

If an upper space keeping isolation member and a lower space keeping isolation member of the same insulating dielectric plate are set up in the same straight line, the adsorbed dirt will accumulate at a maximum speed at the four right angles and on the windward side of the insulating dielectric plate, and then the resistance of the leakage path with another insulating dielectric plate will be reduced at the fastest speed, and the leakage current will be increased. The staggered space keeping isolation member of the present application undoubtedly minimizes the leakage path within a defined space.

Through this way, for example, in the commonly used electrostatic purification device inside the air conditioning box, the width of (length of 500 mm, thickness of 50 mm) purification device is 310 mm, a dust collection space is formed at every 2.14 mm thickness, or adjacent high and low potential insulating dielectric plates occupy the dust collection space, there are a total of 144 dust collection spaces, so that it will eliminate 288 possible leakage paths for the leakage current. In some applications, the thickness of the dust collection space will be about 1 mm, and for the same width, the paths for generating leakage currents will be even more, using the design of the present application, the leakage paths will be greatly reduced and the generation of leakage currents will be reduced.

The insulating dielectric plate of this embodiment is provided with electrode materials, which are usually provided on one surface. The electrode material is recessed at a preset distance relative to each of the four edges of the insulating dielectric plate, i.e., at a certain distance from the edge, so as to avoid the electrode from generating a discharge phenomenon with the surrounding items. The recessed distance at one end in the length direction is greater than the recessed distance at another end, and the insulating dielectric plates in an upper layer and a lower layer are stacked horizontally opposite to each other when stacked. As shown in FIG. 5, the insulating dielectric plates of two adjacent layers are stacked in opposite horizontal directions, for example, the electrode material in the upper layer has a larger recessed distance (L2) on the right side and a smaller recessed distance (L1) on the left side, while its adjacent lower layer has a larger recessed distance on the left side and a smaller recessed distance on the right side.

For example, in the length direction, the recessed distance at one end is 10-30 mm, and the recessed distance at another end is 2-10 mm; the recessed distance from the left and right edges is 2-15 mm. The left and right edges of the electrode material retain a certain distance from the edge of the filter cartridge, which prevents the electrode from ignition phenomenon. The length and width of the electrode material are compatible with the filter cartridge design and are not limited here. The electrode material is a composite of resin, conductive material, flame retardant, adhesive and other materials, and has semi-conducting characteristics, and the internal resistance value within the range of 10⁵-10 8 megohm.

It should be noted that any significant leakage current between two groups of insulating dielectric plates in the electrostatic purification device will result in a decrease in the voltage between the plates, resulting in a decrease in the purification efficiency of the particulate matter once. If the high voltage power supply module outputs a constant voltage, then the power consumption will increase as the leakage current increases, and when the leakage current reaches the maximum current of the high voltage power supply, the voltage across the purification device will drop or cause a reduction in the efficiency of the electrostatic purification device. And the increase of the leakage current will cause local ignition, or ablation, to damage the structure of the purification device. Therefore, the present application maximises the reduction of leakage current by recessing the electrode material into the insulating dielectric plate at different distances.

The main leakage current in the electrostatic purification device is along two groups of insulating dielectric plates with different potentials. The leakage current is affected by the voltage between the dielectric plates, the length of the leakage distance, the relative humidity of the working environment, and the electrical conductivity of the surface of the dielectric plates. A clean purification device has no problem that external factors increase the leakage current. When the air needed to be purified pass through the purification device, pollutants and other dirt in the air are adsorbed by the insulating dielectric plate of the purification device to be constantly accumulated, so it will cause the leakage distance between the insulating dielectric plates is reduced, thereby increasing the leakage current; and the insulating dielectric plate accumulates dirt, most of which has a significant electrical conductivity, so it will increase the conductivity of the surface of the insulating dielectric plate, thereby increasing the leakage current; at the same conditions, the increase of the relative humidity will also reduce the resistance generated by the physical distance between two sets of insulating dielectric plates with different potentials, and also increase the generation of leakage current. In order to reduce the generation of leakage current, it can reduce the voltage between the two groups of insulating dielectric plate, but it will reduce the strength of the electric field between the insulating dielectric plate, charged particles pass through the space between the insulating dielectric plates, the force by the electric field which they are subjected to will reduce, so the voltage between the insulating dielectric plates will weaken the adsorption capacity of the particles and other pollutants, which will affect the purification efficiency of the electrostatic purification device.

Therefore, the electrode material of the present application is recessed into the two ends of the insulating dielectric plate at different distances, so that after stacking and melting and cutting the grooves, the insulating dielectric plate with high and low potentials can form a large physical space for isolation, and the possibility of leakage current is greatly reduced, and even if there is some dirt accumulated at the end of the insulating dielectric plate, due to the high and low potentials of the insulating dielectric plate at a longer distance, i.e., the leakage path is long, so the leakage current will not be easily generated.

The present embodiment uses stacking of insulating dielectric plates provided with electrode materials, and the number of layers to be stacked is determined according to the size of the filter cartridge that is ultimately required. By means of different stacking methods, it is possible for the side having the electrode material to be wrapped in the entire filter cartridge, thereby avoiding the exposure of the electrode material. It is also possible to stack and then cover the side with the exposed electrode material with at least one additional layer of insulating dielectric plate with no electrode material, thereby completely sealing the electrode material in the insulating dielectric, or, cover with a layer of other insulating dielectric material, which will not be described herein.

The conventional insulating dielectric plate is not effectively pressed between layers of insulating dielectric plates, and gaps between layers can easily lead to exposed printed circuits, resulting in current leakage and arcing and ignition. The glue is used to paste between layers of insulating dielectric plates, heat and other external influences may lead to poor internal sealing and printed circuit exposed, resulting in current leakage, arcing and ignition. The present application adopts the method of heat wire thermal fusion cutting to cut out the filter cartridge, and each edge of the filter cartridge is cut by the heat wire thermal fusion cutting. When the heat wire thermal fusion cutting is used, the insulating dielectric material on the cut edges is thermally fused and mildly liquefied to form a rolled edge, and the cut edges between the layers intermingle after cooling, to seal the electrode material. The present application does not use the glue pasting method between the layers of the filter cartridge plate, which overcomes the problems of poor internal sealing and exposed printed circuits caused by the traditional processing method, which are prone to current leakage, arcing and ignition, etc., and can realise the insulating dielectric material to be completely wrapped without breakage.

In a specific embodiment, when the ambient temperature is 24-25° C., the heating temperature of the heating wire ranges from 190° C. to 210° C., preferably 200° C. The advancement rate of the heating wire is 5-20 mm/min, preferably 12 mm/min.

The raw materials of the insulating dielectric plate of the present application include granular insulating dielectric raw materials, flame retardant and colour masterbatch, which are firstly subjected to secondary granulation. Before the secondary granulation, the granular insulating dielectric raw material is first dried, and the drying can evaporate the water vapour inside the dielectric material, avoiding air bubbles in the extrusion moulding and affecting the flatness of the surface of the frame of the isolation-carrying electrode material. Specifically, the weight ratios of the granular insulating dielectric raw material, the flame retardant, and the colour masterbatch of the embodiment are 89%-93% of the dried granular insulating dielectric raw material, 3%-8% of the flame retardant, and 1%-3% of the colour masterbatch, respectively.

The granular insulating dielectric raw materials is made of one or more of polyethylene, polypropylene, polyvinyl chloride, polystyrene, acrylonitrile-butadiene-styrene co-polymer, polyformaldehyde, polyamide, polycarbonate, polyethylene terephthalate, or polyimide. The flame retardant is one or more of triisocyanate (2,3-dibromopropyl), tribromophenol, trimethylbenzene phosphate, triphenyl phosphite, or dimethyl methylphosphonate. The colour masterbatch corresponds to the selected granulated insulating dielectric raw material.

The raw material after secondary granulation has a melt index of 3-6 g/10 min. Too low melt index will result in the hot melt cut quickly turning into a liquid, and too high melt index will result in the melt cut not being able to realize its rolled edges to fully encapsulate the internal electrode material.

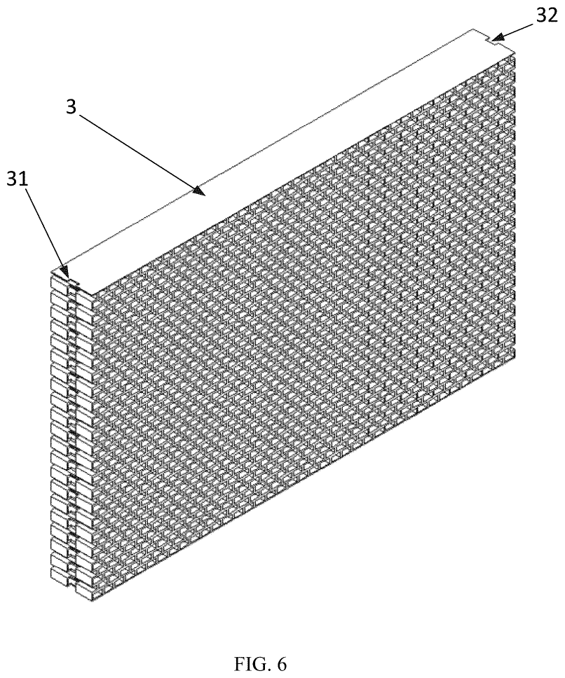

As shown in FIG. 6, two electrode strip holding slots, i.e. a first groove 31 and a second groove 32, are cut by hot fusion at each end of the cartridge 3 in the length direction, the electrode strip holding slots are perpendicular to the plane where the electrode material is located. The electrode strips have an electrically conductive function, and the material is not limited herein.

Since the electrode materials on the insulating dielectric plate are recessed at different distances at two ends in the length direction, after stacking, there is bound to be a part of the electrode materials on the side of the first groove 31 and close to the first groove 31, and another part of the electrode materials is far away from the first groove 31, and the part of the electrode materials far away from the first groove 31 and on the side of the second groove 32 is close to the second groove 32.

In this way, the first electrode strip 71 and the second electrode strip 72, after being loaded into the first groove 31 and the second groove 32, are electrically coupled to the near electrode material to form the positive and negative electrodes of the micro electrostatic purification device, respectively.

In some embodiments, a distance from the bottom of the electrode strip holding slot to the electrode material with a short recessed distance at the end, or to extend into the electrode material with a short recessed distance at the end is 0-10 mm, and the width of the bottom of the electrode strip holding slot is 2-10 mm; the two electrode strip holding slots described are fit with the first electrode strip 71 and the second electrode strip 72 so as to form an independent sealing space, which is sealed by filling the glue, and the electrode strips and their connectors are sealed in the electrode strip holding slots to achieve a better sealing effect.

In this embodiment, the coupling between the electrode strip and the electrode material is electric induction, and the electrode strip holding slot is cut by the hot fusion when cutting, and the depth of the cutting makes the bottom of the holding slot and the electrode material with a short recessed distance at an end where the holding slot is located just contact, or to extend into the electrode material with a short recessed distance at an end where the holding slot is located about 0-10 mm, such that the loaded electrode strip and the electrode material have good electric induction, with a good charged effect.

As shown in FIG. 2, the first electrode strip 71 and the second electrode strip 72 are also connected with insulating wires, and the first groove 31 and the second groove 32 are sealed with sealant 91, 92, so that the first electrode strip 71, the second electrode strip 712 and the connectors with the connected insulating wires, are sealed inside the electrode strip holding slots of the filter cartridge 3, thus realizing complete sealing.

Another end of the insulating wire connected to the first electrode strip 71 and second electrode strip 72 is connected to a power supply module, and the power supply module is an external power supply module; the power supply module includes a sampling and comparison circuit, a current limitation circuit, and a voltage adjustment circuit; when the sampling and comparison circuit detects a change in an output voltage, the voltage adjustment circuit adjusts the output voltage to a constant output voltage; when the sampling and comparison circuit detects that a change in a current exceeds a preset threshold, the current limitation circuit limits a load current.

In this embodiment, the power supply module is external, and the output of the power supply module is connected to the electrode contact piece of the device for power supply, and the electrode contact piece is connected to the electrode strip through insulating wires. Since the power supply module is external, thus another end of the insulating wire is connected to the first electrode strip 41 and the second electrode strip 42, and the first electrode strip 41 and the second electrode strip 42 are connected to the external power supply through the insulating wire.

Specifically, in this embodiment, the first electrode contact piece 41 connecting to the power supply module is connected to the first electrode strip 71 via the first insulating wire 5; and the second electrode contact piece 42 is connected to the second electrode strip 72 via the second insulating wire 6. The first electrode strip 71 and the second electrode strip 72 are provided inside the electrode strip holding slots (the first groove 31 and the second groove 32), and then the two electrode strip holding slots are sealed with sealants 91 and 92.

As shown in FIG. 1, the lower housing 2 is provided with a first chamber 21 and a second chamber 22 at one end corresponding to the first groove 31 to accommodate the first electrode strip 41 and the second electrode strip 42, respectively, and the first chamber 21 and the second chamber 22 are sealed by filling glue after accommodating the first electrode strip 41 and the second electrode strip 42.

In the present application, the electrode materials in the filter cartridge, the electrode contact pieces, the electrode strips, and their connecting parts are all completely sealed by insulating dielectric materials or sealant to achieve sealed insulation of the electrodes, which makes it possible to carry out work smoothly even under high humidity environments with a relative humidity of >80%.

The present application solves the problem that the leakage current increases caused by the sealing problem, two ends of the insulating dielectric plate are cut out grooves by a heating wire, with the depth of the grooves at one end reaching exactly the part of the outer edge of the electrode material that has a shorter recessed distance at this end and the depth of the grooves at another end reaching exactly the part of the outer edge of the electrode material that has a shorter recessed distance at this end, and the upper and lower end surfaces of the frame body carrying the electrode material in the three surfaces of the grooves are sealed by heat fusing, to prevent the infiltration of water, particles, and other dirt in the air along the airflow direction, and to keep the recess and the airflow channel separated from each other, and the recess is in a separate isolation space.

The electrode strip is embedded in the groove to which it is adapted, and then the insulating sealant is potted to securely fix the electrode strip in the groove to prevent current leakage from the electrode strip in the groove. And one of the ends of the electrode strip is connected to the direct current (DC) high-voltage output wire of the high-voltage power supply module to form the high-voltage of the purification device, using the induced voltage of the high-voltage on the electrode material, the electrode strip is conductive with the electrode material and form the required voltage between the successive stacks of insulating dielectric plates.

This design minimises the generation of the leakage current by sealing the electrode material inside the insulating dielectric plates, and sealing the electrode strips connected to the high voltage inside the grooves, the electrode material and the electrode strip are electrically connected by using high voltage induction distinguished from the direct connection of the related art, which helps reducing the path of generation of the leakage current.

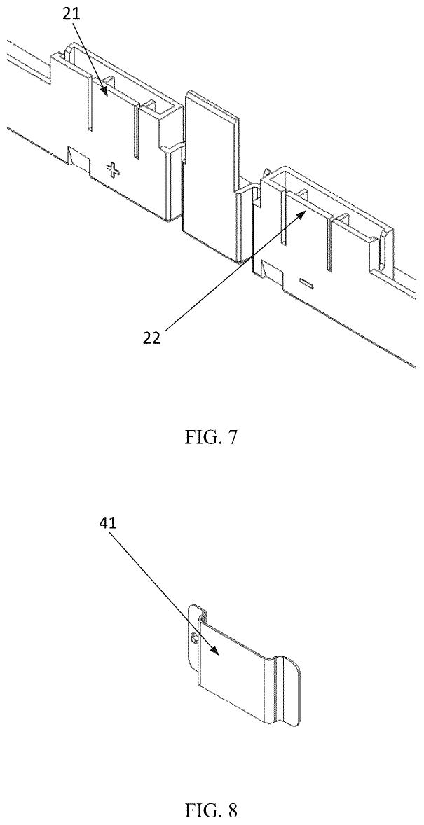

In another embodiment, as shown in FIGS. 7 and 8, two semi-protruding separator plates are provided inside the first chamber 21 and the second chamber 22, respectively, and the centers of the first electrode contact piece 41 and the second electrode contact piece 42 are recessed inwardly and stuck between the separator plates.

In this embodiment, the electrode contact pieces are stuck between the separator plate plates, which form a labyrinth inside the first chamber 21 and the second chamber 22, and are then sealed by filling sealant. The labyrinth sealing structure design is used to seal the bare electrodes with sealant; any bare electrical contact in the circuit are sealed with the sealant, so as to realize the waterproof design on the circuit. The electrode contact pieces are tightly combined with the insulating lower housing 2, and the insulating sealant in the molten state is installed in the first and second chambers, and the electrode contact pieces are completely submerged in the sealant to achieve electrode sealing and insulation. The wire 5 or 6 extends out along the grooves next to the first chamber 21 and the second chamber 22 to connect with the first electrode strip 71 and the second electrode strip 72.

In another specific embodiment, the lower housing 2, as shown in FIG. 7, is provided with two grooves between the first chamber 21 and the second chamber 22. It is possible to increase the creepage distance in case of water or dust.

The power supply module in the first embodiment includes a sampling and comparison circuit, a current limitation circuit and a voltage adjustment circuit, when the sampling and comparison circuit detects a change in the output voltage, the voltage adjustment circuit adjusts the output voltage to a constant output voltage, and when the sampling and comparison circuit detects that a change in the current exceeds a preset threshold, the current limitation circuit limits the load current.

The sampling and comparison circuit detects the output voltage and current, and when a change in the output voltage is detected, the voltage adjustment circuit adjusts the output voltage to a constant output voltage, so that when the load changes, the voltage is automatically adjusted to be consistent with the preset value, and the voltage is output at a constant steady state, such that it keeps high purification efficiency under different humidity environments. When a change in the current is detected over the preset threshold, the current limitation circuit is added to the loop, and the load current is limited by the current limitation circuit, so that when the instantaneous change of the working current exceeds the threshold, current limiting protection is carried out, and when there is an abnormal change in the load, which leads to a large instantaneous change of the working current or the change of the working current exceeding the rated value, the current limitation circuit is automatically cut into the current limitation circuit (about 100 megohm current limitation resistance; different filter cartridges will be designed with different matching resistances), limiting the current surge and upper limit value, such that the filter is kept from ignition, with a good security and reliability, under high humidity environment.

In the previous embodiment, power is supplied through an external power supply module. The power supply module of the present application can also be provided inside the housing of the micro electrostatic purification device for resisting high humidity and powered by an external alternating current (AC) power supply.

In the present application, when the upper and lower housings are fixed in the same filter cartridge, a layer of hot melt adhesive is coated around their insides, or a layer of hot melt adhesive is coated along the windward-side edge and the leeward-side edge of the filter cartridge to increase the firmness and sealing of the connection between the housing and the filter cartridge. Insulating dielectric plates of the same width can also be bonded around the ends of the filter cartridge in the length direction to achieve better sealing.

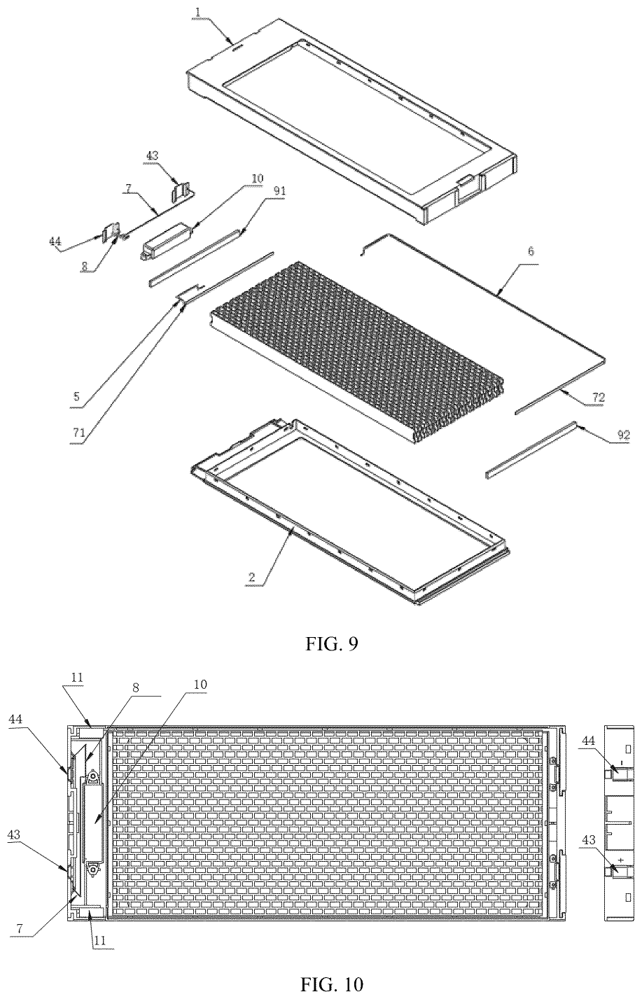

In the second embodiment, as shown in FIG. 9 and FIG. 10, the power supply module 10 of this embodiment is provided inside the housing and is connected to an external power supply through electrode contact pieces.

In this embodiment, the third electrode contact piece 43 and the fourth electrode contact piece 44 are connected to the input of the power supply module 10 through insulating wires 7 and 8, and the first electrode strip 71 and the second electrode strip 72 are connected to the output of the power supply module 10 through insulating wires 5 and 6.

The first electrode strip 71 and the second electrode strip 72 are sealed by filling sealant (91 and 92) after the insulating wires are connected. The connection of the power supply module to the insulating wires is similarly sealed by sealant.

Similarly, the housing (upper or lower housing) is provided with a chamber for mounting the electrode contact pieces, after the electrode contact pieces are connected to the insulating wires, they are placed in the chamber and sealed by sealant.

Both sides of the upper housing 1 or the lower housing 2 of the present embodiment is also provided with magnets 11, and the magnets act with the chassis outside the micro electrostatic air purifier to be able to mount the micro electrostatic air purifier stably in the chassis.

It is to be noted that the mounting location of the electrode contact pieces and the power supply module in the present application can be in the upper housing or in the lower housing, and the above embodiment is only a specific way of implementation, and the present application is not limited to a specific housing structure.

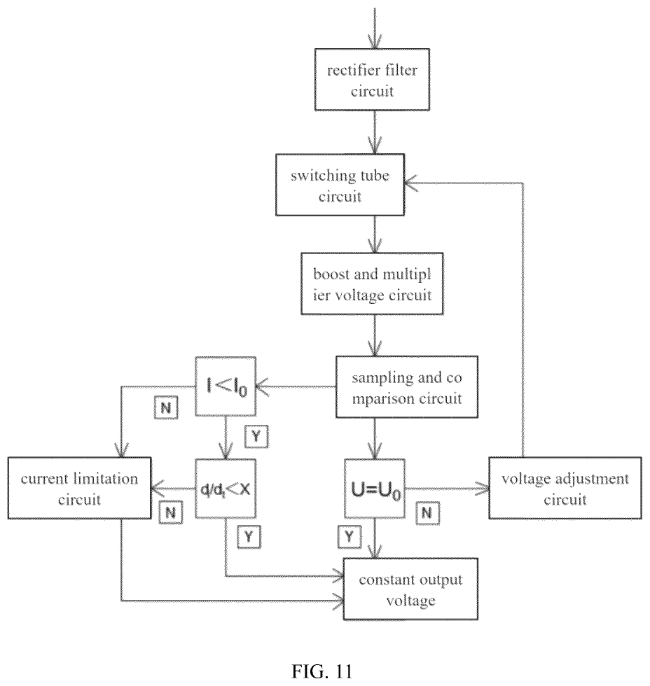

As shown in FIG. 11, the power supply module of the present application includes a rectifier filter circuit, a switching tube circuit, a boost and multiplier voltage circuit, a sampling and comparison circuit, a voltage adjustment circuit, and a current limitation circuit.

The sampling and comparison circuit detects a change in the output voltage, the voltage adjustment circuit adjusts the output voltage to a constant output voltage; and the sampling and comparison circuit detects that a change in the current exceeds a preset threshold value, the current limitation circuit limits the load current.

During operation, the power supply module output a constant voltage to maintain the ionisation intensity, without affection by the loaded micro electrostatic filter and the ambient humidity, so as to ensure the ability and efficiency of the micro electrostatic purification device to adsorb particles. The output voltage U is obtained through the sampling and comparison circuit, and compared with the reference preset voltage U₀ in real time, when it is higher or lower than the preset voltage U₀, and then it is adjusted through the voltage adjustment circuit, to a constant output voltage at a steady state. The power supply module, when in the normal operating state, outputs a constant voltage. if the environmental humidity becomes high, the air becomes more easily ionised, or with the accumulation of running time, the dust in the microporous channel in the micro electrostatic filter becomes more, resulting in that the space between two plates (a plate receives the high voltage and a plate connects to the grounding), becomes smaller, then the current of the power supply module will continue to increase or even sharply increased. Therefore, the output current I is obtained through the sampling and comparison circuit, and when the current transiently surges (di/dt>a certain preset value X) or exceeds a certain rated value Io, it will automatically (nanoseconds) be cut into the current limitation protection circuit, and the current limitation resistance (about 100 megohms) will be added into the constant output voltage circuit. Since the total resistance of the whole circuit increases instantaneously, the output current will become smaller immediately, thus limiting the increase of the current and protecting the micro electrostatic filter from being burnt by high current.

The rectifier filter circuit, the switching tube circuit, the boost and multiplier voltage circuit, the sampling and comparison circuit, the voltage adjustment circuit, and the current limitation circuit in the power supply module of the present application can be implemented by circuits commonly used in the field as far as the individual circuits are concerned, and will not be repeated here.

The above embodiments express only several embodiments of the present application, which are described in a more specific and detailed manner, but are not to be construed as a limitation of the patent application. It should be pointed out that, for those skilled in the art, several deformations and improvements can be made without departing from the conception of the present application, which all fall within the scope of the present application. Therefore, the scope of the patent application shall be subject to the appended claims.

What is claimed is:

1. A micro electrostatic purification device for resisting high humidity, comprising a filter cartridge (3),
wherein the filter cartridge (3) is formed by stacking insulating dielectric plates with an air channel between an upper plate and a lower plate of the insulating dielectric plate, and a space keeping isolation member (102) is provided between the upper plate and the lower plate of the insulating dielectric plate, and the space keeping isolation member (102) and the upper plate and the lower plate of the insulating dielectric plate form the air channel;
an electrode material is provided on the insulating dielectric plate, the electrode material is recessed into a preset distance relative to each of four edges of the insulating dielectric plate, and the recessed distance at one end is greater than the recessed distance at another end in a length direction;
when the insulating dielectric plates with the electrode material are stacked, the insulating dielectric plates in an upper layer and a lower layer are stacked horizontally opposite to each other;
edges of the filter cartridge (3) are cut by a hot fusion to completely seal the electrode material;
two ends of the filter cartridge (3) are cut out by the hot fusion two electrode strip holding slots in the length direction for holding a first electrode strip (71) and a second electrode strip (72) respectively, and the first electrode strip (71) and second electrode strip (72) are electrically coupled to the electrode material with a short recessed distance at an end where the first electrode strip (71) or second electrode strip (72) is located, respectively; and
the first electrode strip (71) and the second electrode strip (72) are also connected with insulating wires respectively, the first electrode strip (71) and second electrode strip (72) and a connector connected with the insulating wires are sealed in the electrode strip holding slot of the filter cartridge (3).

2. The micro electrostatic purification device for resisting high humidity according to claim 1, further comprising an upper housing (1) and a lower housing (2) for accommodating the filter cartridge (3).

3. The micro electrostatic purification device for resisting high humidity according to claim 2, wherein another end of the insulating wire connected to the first electrode strip (71) and the second electrode strip (72) is connected to a power supply module, and the power supply module is an external power supply module;
wherein the power supply module comprises a sampling and comparison circuit, a current limitation circuit, and a voltage adjustment circuit;
when the sampling and comparison circuit detects a change in an output voltage, the voltage adjustment circuit adjusts the output voltage to a constant output voltage;
when the sampling and comparison circuit detects that a change in a current exceeds a preset threshold, the current limitation circuit limits a load current; and
the micro electrostatic filter further comprises a first electrode contact piece (41) and a second electrode contact piece (42) connected to the power supply module, the first electrode contact piece (41) is connected to the first electrode strip (71) through the insulating wire, and the second electrode contact piece (42) is connected to the second electrode strip (72) through the insulating wire.

4. The micro electrostatic purification device for resisting high humidity according to claim 3, wherein the lower housing (2) is provided with a first chamber (21) and a second chamber (22) for accommodating the first electrode contact piece (41) and the second electrode contact piece (42), respectively, and the first chamber (21) and second chamber (22) are sealed by filling glue after accommodating the first electrode contact piece (41) and the second electrode contact piece (42).

5. The micro electrostatic purification device for resisting high humidity according to claim 4, wherein each of the first chamber (21) and the second chamber (22) is provided with two semi-projecting separator plates inside, and centers of the first electrode contact piece (41) and the second electrode contact piece (42) are recessed and are stuck between the two semi-projecting separator plates.

6. The micro electrostatic purification device for resisting high humidity according to claim 4, wherein the lower housing (2) is provided with two grooves between the first chamber (21) and the second chamber (22).

7. The micro electrostatic purification device for resisting high humidity according to claim 2, wherein another end of the insulating wire connected to the first electrode strip (71) and the second electrode strip (72) is connected to a power supply module, and the power supply module is provided inside the upper housing (1) or the lower housing (2);
wherein the power supply module comprises a sampling and comparison circuit, a current limitation circuit, and a voltage adjustment circuit;
when the sampling and comparison circuit detects a change in an output voltage, the voltage adjustment circuit adjusts the output voltage to a constant output voltage;
when the sampling and comparison circuit detects that a change in a current exceeds a preset threshold, the current limitation circuit limits a load current;
an output of the power supply module is connected to the first electrode strip (71) and the second electrode strip (72) through the insulating wire, an input of the power supply module is connected to a third electrode contact piece and a fourth electrode contact piece, the third electrode contact piece and the fourth electrode contact piece are clamped in the chamber opened in the upper housing (1) or the lower housing (2); and
a joint where the third electrode contact piece and the fourth electrode contact piece are connected with the insulating wire, and a joint where the power supply module is connected with the insulating wire, are sealed by pouring in a sealant.

8. The micro electrostatic purification device for resisting high humidity according to claim 2, wherein magnets (11) are also provided on both sides of the upper housing (1) or the lower housing (2).

9. The micro electrostatic purification device for resisting high humidity according to claim 1, wherein the air channel of insulating dielectric plates of the filter cartridge (3) are quadrilateral, the quadrilateral air channels are staggered, and the space keeping isolation member of the quadrilateral air channels of one insulating dielectric plate is located between two space keeping isolation members of the quadrilateral air channels of another insulating dielectric plate.

10. The micro electrostatic purification device for resisting high humidity according to claim 1, wherein the electrode material is recessed a preset distance with respect to each of the four edges of the insulating dielectric plate, and the recessed distance at one end in the length direction is greater than the recessed distance at another end, wherein the recessed distance at one end in the length direction is 10-30 mm and the recessed distance at another end is 2-10 mm; and
the recessed distance from the left edge and the right edge is 2-15 mm.

11. The micro electrostatic purification device for resisting high humidity according to claim 1, wherein a distance from a bottom of the electrode strip holding slot to the electrode material with the short recessed distance at the end or extend into the electrode material with the short recessed distance at the end is 0-10 mm, and a width of the bottom of the electrode strip holding slot is 2-10 mm; the two electrode strip holding slots are fit with the first electrode strip (71) and the second electrode strip (72).

12. The micro electrostatic purification device for resisting high humidity according to claim 1, wherein the raw material of the insulating dielectric plate comprises granular insulating dielectric raw material of 89-93% by weight, a flame retardant of 3-8% by weight, and a masterbatch of 1-3% by weight; the granular insulating dielectric raw material is at least one of polyethylene, polypropylene, polyvinyl chloride, polystyrene, acrylonitrile-butadiene-styrene co-polymer, polyformaldehyde, polyamide, polycarbonate, polyethylene terephthalate, or polyimide; the flame retardant is at least one of triisocyanate (2,3-dibromopropyl), tribromophenol, trityl phosphate, trityl phosphite, or dimethyl methyl phosphonate.

13. The micro electrostatic purification device for resisting high humidity according to claim 1, wherein the insulating dielectric plates provided with the electrode materials are stacked and then covered with at least one more layer of insulating dielectric plates not provided with electrode materials on a side where the electrode materials are bare.
